(19) United States
(12) Patent Application Publication
(10) Pub. No.: US 2020/0400674 A1
(43) Pub. Date: Dec. 24, 2020

(54) A METHOD OF DETECTING MAGEA4

(71) Applicant: IMMUNOCORE LIMITED, Abingdon, Oxfordshire (GB)

(72) Inventor: Amanda WILLIAMS, Abingdon (GB)

(21) Appl. No.: 16/979,146

(22) PCT Filed: Mar. 7, 2019
(86) PCT No.: PCT/GB2019/050640

§ 371 Date: Sep. 8, 2020

(30) Foreign Application Priority Data
Mar. 8, 2018 (GB) 1803750.7

(51) Int. Cl. G01N 33/574

(52) U.S. Cl. CPC G01N 33/57484

(57) ABSTRACT

The present invention provides a method of detecting MAGEA4 in a sample. The method may comprise adding an anti-MAGEA4 antibody to the sample at a concentration in the range of from 2-20 μg/ml, incubating the antibody and the sample and detecting antibody that is bound to the sample. The methods of the invention can be used to diagnose whether a subject has cancer and whether the subject is eligible for treatment with a MAGEA4 targeted cancer therapy.

The present invention relates to methods for detecting Melanoma-Associated Antigen A4 (MAGEA4). The methods of the invention can be used to diagnose whether a subject has cancer and whether the subject is eligible for treatment with a MAGEA4 targeted cancer therapy.

MAGEA4 (NCBI Reference Sequence: NP_001011550.1) is a tumour-associated antigen (TAA) belonging to the MAGE family of germline encoded cancer antigens (De Plaen, et al., (1994), Immunogenetics 40(5): 360-369) and is an ideal target for therapeutic intervention. High level expression of MAGEA4 has been reported in tumours of several types including melanoma, carcinomas of the esophagus, the head and neck, the lung, the breast and the bladder (Bergeron, (2009), Int J Cancer 125(6): 1365-1371; Cabezon, et al., (2013), Mol Cell Proteomics 12(2): 381-394; Cuffel, et al., (2011), Int J Cancer 128(11): 2625-2634; Forghanifard, et al., (2011), Cancer Biol Ther 12(3): 191-197; Karimi, et al., (2012), Clin Lung Cancer 13(3): 214-219; Svobodova, et al., (2011), Eur J Cancer 47(3): 460-469). Expression of MAGE A4 in normal tissues is limited to adult testes and other immune-privileged sites including placenta. Thus, detection of MAGEA4 in other tissues is indicative of cancer and can be used to suggest a targeted cancer therapy.

Targeted cancer therapies are designed to specifically interact with molecules involved in the growth, progression or spread of cancer. In recent years, the number of such treatments that are approved or in clinical testing has risen substantially. Concurrent with this rise has been an increased requirement for suitable companion diagnostic methods that are able to identify those patients who are most likely to benefit from a particular therapy.

For therapies that are designed to specifically recognise tumour-associated antigens (TAAs) such as MAGEA4, detecting expression of a given TAA in patient tumour samples provides a convenient diagnostic approach to classify patients as likely responders and therefore eligible for treatment with the corresponding therapy. While there are a number of known techniques that may be applied to the detection of TAAs, obtaining an accurate and reproducible companion diagnostic assay is not straightforward and requires careful optimisation. False negative results could mean that a patient is not given a potentially beneficial treatment, while false positive results could lead to unnecessary, and potentially toxic, side-effects for the patient. Therefore, there remains a continuing need for diagnostic methods to detect TAAs, diagnose cancer and identify eligible patients prior to treatment.

Immunohistochemistry (IHC) is a well-known diagnostic method. IHC may be performed using an automated staining system. Such a system enables standardisation and increased reproducibility. Automated staining systems can be obtained commercially from a number of vendors including Ventana/Roche, Dako/Agilent, Leica ThemoFisher. Unlike other methods, such as PCR, IHC can provide information of cellular localisation as well as expression level. This is particularly important for tumour samples, where antigen expression can be heterogeneous. The FDA-approved Herceptest (Agilent-Dako) is considered the prototype IHC-based companion diagnostic assay for targeted therapies. In this case, a positive test classifies a patient as a ‘responder’ to the Her2 targeted antibody trastuzumab (Herceptin) for breast cancer. A more recent example is the IHC diagnostic assay for PD-L1 antibody pembrolizumab in non-small-cell lung cancer (Roach et al., (2016), Appl. Immunohistochem. Mol. Morphol. 24(6): 392-397). Despite the broad acceptance of IHC as a diagnostic method, there remains issue with reproducibility, a lack of standardisation and a plethora of reagents, detection systems and assay conditions (Taylor et al., (2014), Appl. Immunohistochem. Mol. Morphol. 22(8): 555-561), all of which makes the development of a diagnostic assay far from routine.

In a first aspect, the present invention provides a method of detecting MAGEA4 in a sample, the method comprising:
adding an anti-MAGEA4 antibody to the sample at a concentration in the range of from 2-20 μg/ml;
incubating the antibody and the sample; and
detecting antibody that is bound to the sample.

The inventors have found that the particular concentration of antibody allows accurate and reproducible detection of MAGEA4, thereby minimising false positive and false negative results.

The anti-MAGEA4 (or “primary” antibody) may be a monoclonal antibody. It may be a human or mouse antibody and/or may be an IgA, IgD, IgE, IgG or IgM subtype. As is known to those skilled in the art, MAGEA4 shares high level of sequence homology to other proteins of the MAGE family. Therefore, to minimise false positives, it is preferable that the primary antibody is able specifically to recognise MAGEA4, and demonstrates limited or no cross reactivity with other MAGE family members, when assessed in tumour samples.

As used herein, the term “antibody” refers to an immunoglobulin that specifically binds to, and is thereby defined as complementary with, a particular spatial and polar organisation of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained. The terms “monoclonal antibody,” “mAb” and “MAB” refer to an antibody that is an immunoglobulin produced by a single clone of lymphocytes which recognises only a single epitope on an antigen. For example, a monoclonal antibody useful for the methods disclosed herein displays a single binding specificity and affinity for a particular epitope of MAGEA4. The term “polyclonal antibody” as used herein refers to a composition of different antibody molecules which is capable of binding to or reacting with several different specific antigenic determinants on the same or on different antigens. The variability in antigen specificity of a polyclonal antibody is located in the variable regions of the individual antibodies constituting the polyclonal antibody, in particular in the complementarity determining regions (CDRs).

A preferred anti-MAGEA4 antibody is OTI1F9, which is a mouse IgG2a monoclonal antibody commercially available from Origene (Cat #TA505362). Other primary antibodies may be used, including CPTC-MAGEa4-1 (Developmental Studies Hybridoma Bank—DSHB Cat #CPTC-MAGEA4-1, RRID:AB_2138142).

The concentration of the antibody may be in the range of from 4-15, 4-10, 5-13, 6-12, 7-11 or 6, 7, 8, 9, 10, 11 or 12 μg/ml. A preferred range is 4-10 and a preferred concentration is 10 μg/ml. In those embodiments of the invention that use the OTI1F9 antibody, the concentration used may be 6-12, 7-11 or 9, 10 or 11 μg/ml, with 10 μg/ml being preferred. The conditions and duration of incubation will depend on the particular antibody used. The sample and the antibody may be incubated for 30-60, 25-50, 25-39, 27-35, 28-34, 30-33, 31, 32 or 33 minutes at 36° C. The sample and the antibody may be incubated for any of the above times at 33-39, 34-38, 35-37, 35, 36 or 37° C. The inventors have found that 32 minutes at 36° C. provides optimal results, particularly for OTI1F9. However, the time and temperature may be varied. For example, if a lower temperature is used, the time of incubation may be longer, e.g. 4° C. overnight. Equally if a higher temperature is used, the time of incubation may be shorter.

The anti-MAGEA4 antibody that is bound to the sample may be detected by a number of techniques. Detection may be performed directly or indirectly. In direct detection, binding of antibody to MAGEA4 is determined directly, using a labelled reagent, such as a primary antibody labelled with a fluorescent-tag, enzyme or chromogenic or fluorogenic substrate, which can be visualised without further antibody interaction. The antibody may be conjugated to an enzyme, such as peroxidase, that can catalyse a colour-producing reaction. Alternatively, the antibody can also be tagged to a fluorophore thus employing the principles of immunofluorescence. In indirect detection, unconjugated primary antibody binds to MAGEA4 and then a labelled secondary antibody binds to the primary antibody. Suitable secondary antibodies may be raised against the antibody isotype of the animal species in which the primary antibody has been raised. For example, the secondary antibody may be an anti-mouse antibody, capable of binding to mouse antibodies. Methods using a secondary antibody may be more sensitive than direct detection methods, due to signal amplification from multiple secondary antibodies binding the primary antibody. The secondary antibody may be conjugated to an enzymatic label, a chromogenic, or fluorogenic substrate to provide visualisation of the antigen. Suitable labels include enzymes such as horseradish peroxidase, alkaline phosphatase, glucose oxidase and luciferase, and colourimetric agents, including quantum dots, fluorophores and chromophores. Visualisation reagents may be provided as part of a kit such as those commercially available from Dako/Agilent and Ventana/Roche. The method of the invention is preferably an indirect assay.

The secondary antibody may be attached to any suitable fluorophore. In certain cases, a fluorophore may be a coumarin, a cyanine, a benzofuran, a quinoline, a quinazolinone, an indole, a benzazole, a borapolyazaindacene and or a xanthene including fluorescein, rhodamine and rhodol.

Specific fluorescent dyes of interest include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G, 6-carboxyrhodamine-6G, and rhodamine 110; cyanine dyes, e.g., Cy3, Cy5 and Cy7 dyes; coumarins, e.g., umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, Napthofluorescein, Texas Red, Cy3, and Cy5, etc.

Suitable distinguishable fluorescent label pairs useful in the invention include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002), Ried et al. (Proc. Natl. Acad. Sci. 1992: 89: 1388-1392) and Tanke et al. (Eur. J. Hum. Genet. 1999 7:2-11) and others.

Antibody binding can be viewed with a fluorescence microscope and an appropriate filter for each fluorophore, or by using dual or triple band-pass filter sets to observe multiple fluorophores—see e.g., U.S. Pat. No. 5,776,688.

A variety of detectable enzymatic substrates are available for use with enzymatically labelled antibodies. These include chromogenic substrates, such as pNNP, BCIP/NBT (5-bromo-4-chloro-3′-indolyphosphate/nitro-blue tetrazolium), TMB (tetramethybenzidine), DAB (3,3′-diaminobenzidine), OPD (ortho-phenylenediaine dihydrochloride) and ABTS (2,2′-azinobis[-ethylbenzothiazoline-6-sulfonic acid]), and chemiluminscent substrates such as an ECL (enhanced chemiluminscent) label or Acridinium ester (AE). Antibody binding can be viewed with a light microscope. OTI1F9 may be detected using a secondary anti-mouse antibody that may be conjugated to horseradish peroxidase (HRP) or to a fluorophore such as Alexa Fluor® Plus 488. Such secondary antibodies are known in the art.

The secondary antibody may preferably be conjugated to HRP, and comprise a cocktail of HRP labelled antibodies (goat anti-mouse IgG, goat anti-mouse IgM, and goat anti-rabbit). The anti-MAGEA4 antibody/secondary antibody complex may preferably be visualized with hydrogen peroxide substrate and 3, 3′-diaminobenzidine tetrahydrochloride (DAB). The hydrogen peroxide substrate may be 0.4% and 3, 3′-diaminobenzidine tetrahydrochloride (DAB) chromogen may be 0.2%. Copper sulphate (5 g/L) in an acetate buffer is preferably used to enhance staining. Staining may be assessed by light microscopy.

Detection kits provided by Ventana/Roche may be used with the timings as instructed. Other detection kits could be used. Preferably the anti-MAGEA4 antibody (e.g. OTI1F9) is detected using the ultraView detection kit supplied by Ventana Medical Systems (product code 760-500), incubating the slides in HRP-multimer for 20 mins at 36° C. In this instance, the appearance of a brown precipitate indicates the presence of MAGEA4, and that the subject may be eligible for treatment with a MAGE-A4 targeted therapy.

Staining may be compared to a control. Controls are useful to support the validity of staining, and to identify experimental artefacts. In some cases, the control may be a reference sample or reference dataset. The reference may be a sample that has been previously obtained from a subject with a known degree of suitability. The reference may be a dataset obtained from analysing a reference sample. Controls may be positive controls in which the target molecule is known to be present, or expressed at high level, or negative controls in which the target molecule is known to be absent or expressed at low level. A suitable positive control tissue is testis and a suitable negative control tissue is ovary.

Controls may be samples of tissue that are from subjects who are known to benefit from the treatment. The tissue may be of the same type as the sample being tested. For example, a sample of tumour tissue from a subject may be compared to a control sample of tumour tissue from a subject who is known to be suitable for the treatment, such as a subject who has previously responded to the treatment. In some cases, the control may be a sample obtained from the same subject as the test sample, but from a tissue known to be healthy. Thus, a sample of cancerous tissue from a subject may be compared to a non-cancerous tissue sample. In some cases, the control is a cell culture sample. In some cases, a test sample is analysed prior to incubation with an antibody to determine the level of background staining inherent to that sample. In some cases, an isotype control is used. Isotype controls use an antibody of the same class as the target specific antibody, but are not immunoreactive with the sample. Such controls are useful for distinguishing non-specific interactions of the target specific antibody.

To ensure accurate interpretation of test results, interpretation of morphology and immunohistochemistry may be carried out by a pathologist. The method may involve confirmation that the pattern of expression correlates with the expected pattern. The method may involve confirmation that the ratio of target signal to noise is above a threshold level, thereby allowing clear discrimination between specific and non-specific background signals.

Detecting MAGEA4 may optionally or additionally include determining the level of MAGEA4 expression in the sample. The level of MAGEA4 may be determined quantitatively or semi-quantitatively. A subject may be determined to be suitable for treatment, or selected for treatment, if the level of MAGEA4 is elevated or overexpressed in the sample. In some cases, the level of MAGEA4 is determined relative to a control.

Ultimately, determination of patient eligibility for treatment will be performed by a qualified pathologist who is experienced in IHC procedures.

The sample used in the method of the present invention may be a cell line, which may be human. Alternatively, and preferably, it is a tissue sample from a subject, which may be human. The sample may be a human tumour tissue sample.

The term “sample,” as used herein, encompasses a variety of sample types obtained from a subject and can be used in a diagnostic or monitoring assay. The sample may be of a healthy tissue, diseased tissue or tissue suspected of being diseased tissue. The sample may be a biopsy taken, for example, during a surgical procedure. The sample may be collected via means of fine needle aspiration, scraping or washing a cavity to collect cells or tissue therefrom. The sample may be of a tumour such as, for example, solid and hematopoietic tumours as well as of neighbouring healthy tissue. The sample may be a smear of individual cells or a tissue section. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses clinical samples, and also includes cells in cell culture, cell supernatants, cell lysates, cell extracts, cell homogenates, and subcellular components including synthesized proteins, serum, plasma, bodily and other biological fluids, and tissue samples. The biological sample can contain compounds that are not naturally intermixed with the cell or tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. The sample may be prepared directly for performance of the method of the invention, alternatively archived samples may be used.

The method of the present invention may additionally include one or more pre-treatment steps prior to addition of the anti-MAGEA4 antibody to the sample.

Pre-treatment may include the step of epitope retrieval, or unmasking, prior to adding the anti-MAGEA4 antibody to the sample. Fixation of tissue by formalin results in the formation of covalent bonds between the aldehyde and amino groups present in the tissue. The formation of these bonds denatures protein and can result in the loss of antigenicity. In addition, the formaldehyde can form methylene bridges cross linking tissue proteins, thus reducing the penetration of the tissue to large molecules such as antibodies. Removing these bonds, using a combination of pH and temperature, allows renaturation of protein molecules and increases antibody accessibility. Often these changes result in significant gains in antibody binding and improved signal to noise ratios. In general, any buffer suitable for epitope retrieval—particularly those supplied by Ventana/Roche—is suitable for this step. The buffer used for this epitope retrieval or unmasking step may be EDTA based (pH 8-9) or citric acid based (pH6). The inventors have found that an EDTA based buffer gives a better cell appearance compared to the citric acid buffer. A suitable buffer may contain 10 mM Tris Base, 1 mM EDTA, 0.05% Tween 20, pH 8.0; such buffers are available commercially, for example cell conditioning fluid 1 supplied by Ventana Medical Systems (product code 950-124). The time and temperature at which this step is carried out may vary provided that the right balance between morphological appearance and destruction of tissue is achieved. A higher temperature generally needs shorter incubation, while a lower temperature generally needs longer incubation. The temperature of the epitope retrieval step may be 80-110, 90-100, 92-108, 94, 95 or 96° C. The duration of the epitope retrieval step may be 10-30, 12-28, 13-27, 14-26, 15-25, 16-24, 17-23, 18-22, 18, 19, 20, 21 or 22 minutes. A preferred epitope retrieval step is performed at 95° C. for 20 mins and a preferred buffer is cell conditioning fluid 1 supplied by Ventana Medical Systems (product code 950-124).

Optionally enzymes such as proteases may also be added in the epitope retrieval step. The protease used may be any suitable protease, including a serine protease, a metallo-protease, and a cysteine protease. The protease used may be selected from the group consisting of trypsin (from, e.g., bovine), chymotrypsin (from, e.g., bovine), endoproteinase Asp-N (from, e.g., Pseudomonas fragi), endoproteinase Arg-C (from, e.g., mouse submaxillary gland and Clostridium histolytcum), endoproteinase Glu-C (from, e.g., Staphylococcus aureus), endoproteinase Lys-C (from, e.g., Lysobacter enzymogenes), pepsin (from, e.g., porcine), thermolysin (from, e.g., Bacillus thermoproteolytcus), elastase, papain (from, e.g., Carica papaya), proteinase K (from, e.g., Tritirachium album), subtilisin (from, e.g., Bacillus subtilis), proteinase K, furin, and ficin.

The method of the present invention optionally comprises one or more of and preferably all of the following pre-treatment steps, particularly where the sample is a tissue sample.

The sample may be treated with a reagent to inhibit endogenous peroxidase activity. Endogenous peroxidases react with hydrogen peroxide to reduce the 3, 3′-diaminobenzidine (DAB) substrate or other peroxidase substrates, resulting in nonspecific staining of the tissue. The most common method for inhibiting endogenous peroxidase activity is incubation of the sample in a solution of hydrogen peroxide. A suitable concentration of hydrogen peroxide may be 3% v/v, alternatively a 0.3% solution may be used if, for example, tissue damage is evident with a higher concentration. Methanol, PBS, distilled water or saline can be used to dilute hydrogen peroxide. This step is particularly preferred where the sample is a tumour sample and where chromogenic detection involves a HRP-labelled antibody. The inhibition of endogenous peroxidases can be performed before or after epitope retrieval or before and after the primary antibody incubation step. In a preferred embodiment, endogenous peroxidases are inhibited subsequent to the epitope retrieval step (where present). In a further preferred embodiment, the inhibitor comprises 3% hydrogen peroxide, and is obtained from a commercial vendor. The inhibitor may be ultraView Universal DAB Inhibitor supplied by Ventana Medical Systems (product code 253-4291). The sample may be exposed to the DAB inhibitor for at least 5, 6, 7, 8, 9, 10 or 11 minutes at room temperature to ensure sufficient inhibition. At least 8 minutes at room temperature is preferred. This pre-treatment step may be up to 60 minutes or up to 15 minutes; 8-15 minutes is preferred.

The method of the invention may further comprise a step of incubation in a blocking reagent. This step can reduce background signal and may be optional where the sample is a cell line. In principle, any protein that does not bind specifically to the target antigen or the antibodies used in the method of the present invention and other detection reagents in the assay can be used for blocking. In practice, however, certain proteins perform better than others, because they bind readily to nonspecific sites (also called reactive sites) at neutral pH or stabilize the function of other assay components. Examples of blocking agents include normal serum, such as goat serum and/or proteins such as albumin, gelatin, casein, or dried milk. There are a wide range of commercial blocking buffers available. In a preferred embodiment, the blocking buffer contains goat globulin and casein. For example, 100 mM phosphate buffer with up to 20% goat globulin and casein. A preferred commercial blocking reagent is Antibody Diluent with Casein supplied by Ventana Medical Systems (product code 760-219). The blocking step for IHC is preferably performed after all other sample pre-treatment steps are completed, and just prior to incubating the sample with the primary antibody. Incubation with the blocking reagent may be for 7-20, 8-16, 9-15, 10-14, 11-13, 11, 12 or 13 minutes at 36° C. Incubation with the blocking reagent may be for any of the above times at 33-39, 34-38, 35-37, 35, 36 or 37° C. The inventors have found that 12 minutes at 36° C. provides optimal results. However, the time and temperature may be varied. For example, if a lower temperature is used, the time of incubation may be longer. For example, incubation with blocking regent may be performed overnight at 4° C. Equally if a higher temperature is used, the time of incubation may be shorter.

As mentioned above, the sample may be preserved as a formaldehyde- or paraformaldehyde-fixed paraffin-embedded (FFPE) tissue preparation. In this instance, the method of the present invention will be carried out on a deparaffinised sample or include an initial step of deparaffinisation. Suitable conditions for deparaffinisation are known to those in the art.

The paraffin wax may be removed by solvent exchange, e.g., exposing the sample to a paraffin solvent such as xylene, toluene or limonene, the solvent then being removed by alcohol, and the alcohol removed by sequential alcohol/water mixtures of decreasing alcoholic concentrations, until eventually the tissue is once more infiltrated by water or aqueous solutions. The infiltration of the sample by water permits the staining of the cell constituents by water soluble chemical and immunochemical dyes.

Toxic paraffin solvents such as xylene and toluene may be replaced with less toxic nonpolar organic solvents such as Terpene Oil (e.g. AmeriClear™, Baxter Healthcare Diagnostics, McGaw Park, Ill.), isoparaffinic hydrocarbons such as MicroClear™ from Micron Diagnostics of Fairfax, Va., and Histolene, a dewaxer that is 96% d-Limonene (Fronine Pty Ltd, Riverstone, New South Wales, Australia). Automated methods may be used. For example, Ventana Medical Systems' U.S. Pat. No. 6,544,798 describes an automated method of removing paraffin wax from tissue sections using only hot water with surfactant. The process relies on the physical partitioning of the liquefied paraffin from the tissue by taking advantage of the immiscibility of liquefied paraffin and hot water. The process is widely used on the BENCHMARK series of automated tissue stainers. A related method based on water and an emulsifying surfactant is known from U.S. Pat. No. 6,649,368. U.S. Pat. No. 6,632,598 (Zhang et al.) describes methods and compositions for deparaffinizing paraffin-embedded tissue. The method involves contacting a paraffin wax-embedded specimen with a dewaxing composition to solubilize the wax impregnating the specimen prior to histochemical analysis. The dewaxing compositions specifically include a paraffin-solubilizing organic solvent selected from the group consisting of aromatic hydrocarbons, terpenes and isoparaffinic hydrocarbons, a polar organic solvent, and a surfactant to solubilize the wax associated with the specimen. Compositions can further comprise water.

A preferred deparaffinisation step comprises heating the sample to 60° C. for 30 minutes to melt the wax and subsequently incubating the sample in a suitable aqueous detergent solution for 3 cycles of 8 minutes at 69° C. A suitable commercial buffer is EZ-Prep supplied by Ventana Medical Systems (product code 950-102). A typical temperature range for initial heating is 50-70° C. and for incubation with reaction buffer is 68-71° C. Processing samples below this temperature range could result in wax remaining in the sample and inefficient epitope access, whilst temperatures above this range could result in tissue destruction. Furthermore, the number of cycles can be increased (to e.g. 4, 5, or 6) or decreased (to e.g. 1 or 2). An increase in cycles may involve a reduction in the length of the cycle (to e.g. 3, 4, 5, 6 or 7 minutes). A decrease in cycles may involve an increase in the length of the cycle (to e.g. 9, 10, 11, 12 or 13 minutes).

A preferred method of the invention comprises the following steps:
deparaffinising the sample, for example by heating the sample to 60° C. for 30 minutes and subsequently incubating the sample in reaction buffer such as EZ-Prep solution, for 3 cycles of 8 minutes at 69° C.;
retrieving epitope, for example by incubating the sample in cell conditioning fluid 1 at 95° C. for 20 mins;
inhibiting endogenous peroxidase activity, for example by incubating the sample in DAB inhibitor for 8 mins at room temperature;
incubating the sample in blocking reagent, optionally for 12 mins at 36° C.; adding antibody OFI1F9 to the sample at a concentration of 10 μg/ml;
incubating the antibody and the sample for 32 mins at 36° C.; and detecting antibody that is bound to the sample.

Detection of antibody may be made using ultraView detection kit as instructed by the manufacturer.

IHC in accordance with the invention may be performed using an automated staining system. Preferably the automated staining system is a Ventana BenchMark ULTRA. Alternative slide staining systems include Ventana BenchMark GX, Ventana BenchMark GT, Dako Autostainer Link 48, Dako Omnis, Leica BOND-Ill and Leica BOND-MAX.

The method of the invention may be used in isolation or may be used in combination with other assays including but not limited to, other morphological stains, in situ hybridisation, qRT-PCT, ELISA, Western blotting, proteomics, FACS.

The method of the invention may be provided as part of a diagnostic kit for determining the presence of MAGEA4 protein in a tumour sample. Preferably the kit is used to determine the eligibility of a patient for a MAGEA4 targeted therapy. The kit may comprise an anti-MAGEA4 antibody in combination, optionally with the reagents necessary for carrying out the method of the present invention.

The kit may be suitable for a point-of-care in vitro diagnostic test. It may be a kit for laboratory-based testing. The kit may include instructions for use, such as an instruction booklet or leaflet. The instructions may include a protocol for performing the method of the invention. The instructions may include a protocol for performing an IHC assay. They may describe methods and suggestions for adapting the test for different types of sample. They may provide methods and suggestions for optimising the results obtained from the test, such as minimising the signal to noise ratio.

The method of the present invention is particularly suited to use as a companion diagnostic to identify eligible patients for a MAGEA4 targeted therapy. Accordingly, in another aspect, the present invention provides a method of treating a human or animal mammalian subject in need thereof—advantageously comprising providing individualised or personalised treatment involving a MAGEA4 targeted therapy—comprising: detecting MAGEA4 in a sample from the subject by adding an anti-MAGEA4 antibody to the sample at a concentration in the range of from 2-20 μg/ml; incubating the antibody and the sample; and detecting antibody that is bound to the sample wherein, if MAGEA4 is detected in the sample, a MAGEA4 targeted therapy is administered to the subject.

Such therapies include soluble biologics, cell therapies and vaccines. A suitable therapy is a bispecific molecule comprising a soluble engineered T cell receptor with high affinity for a HLA-restricted epitope from MAGEA4, fused to a T cell redirecting anti-CD3 antibody fragment. Preferred examples of such bispecific molecules are described in WO2017175006. Examples of other MAGEA4 targeted therapies include, but are not limited to, those being developed by Adaptimmune (WO2017174824, clinical trial no: NCT03132922), Immatics (WO2017158103), Adicet Bio (WO2016199141) and Takara Bio (clinical trial no: NCT02096614).

The method may be used to treat patients having any tumour type known to express MAGEA4, preferably lung (including NSCLC), oesophageal, head and neck, or urothelial/bladder cancer, as well as melanoma. Other tumour types include gastric, ovarian, colorectal and renal.

Preferred features of each aspect of the invention are for each other aspect of the invention mutatis mutandis. The prior art documents referred to herein are incorporated by reference to the fullest extent permitted by law

The present invention will now be described further in the following non-limiting examples. Reference is made to the accompanying drawings in which:

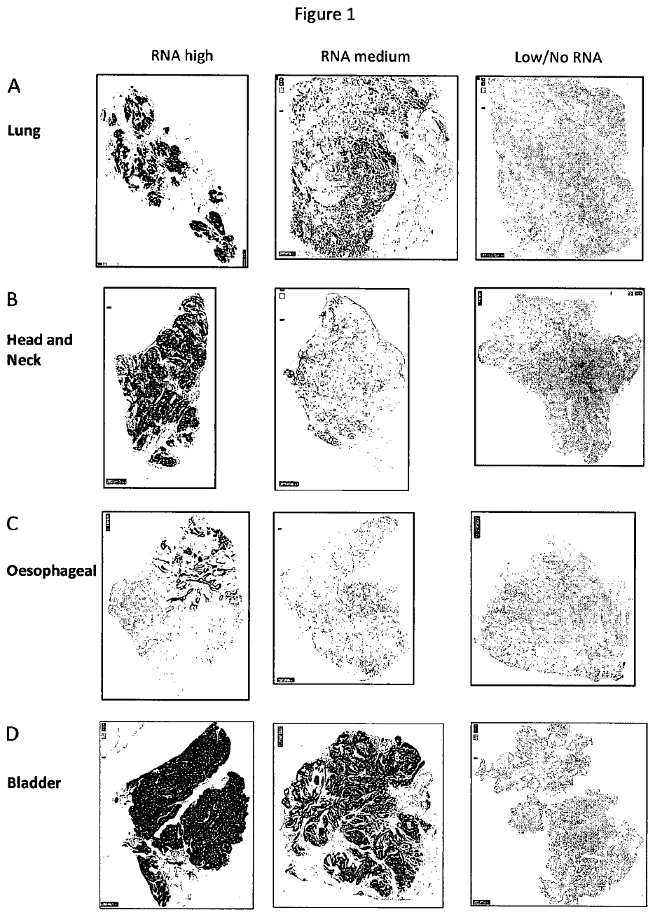

FIG. 1 shows representative staining of samples obtained from lung, oesophageal, head and neck, and bladder cancers (A to D, respectively) in accordance with the invention.

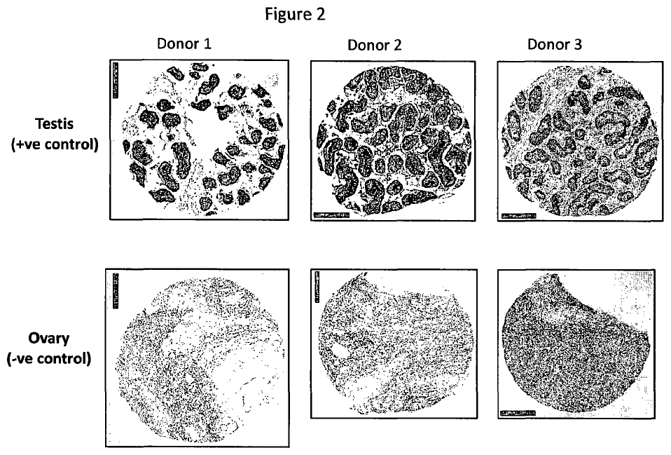

FIG. 2 shows staining of control samples (testis and ovary respectively).

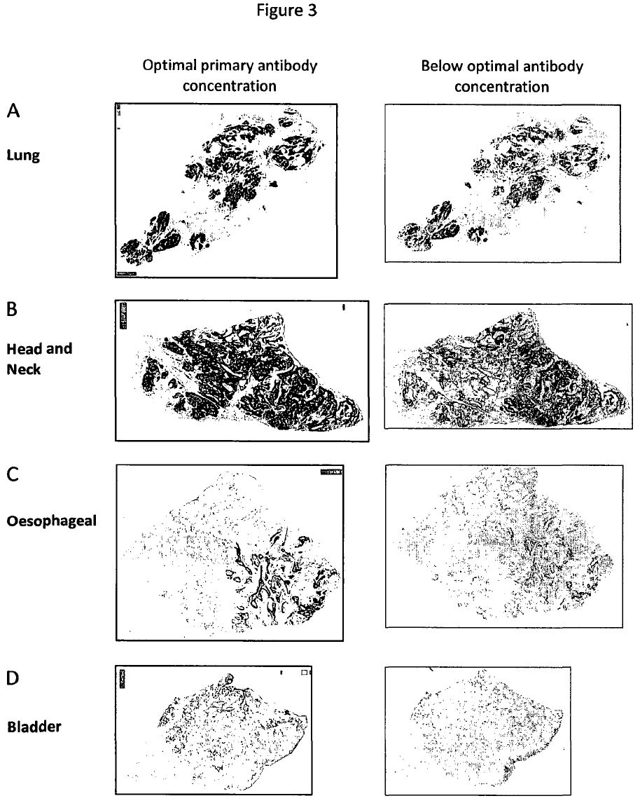

FIG. 3 shows a comparison of staining between optimised and non-optimised antibody concentrations (10 μg/ml and 1.25 μg/ml respectively), for four different tumours (A to D).

### EXAMPLES

### Example 1—IHC Diagnostic Assay for Detection of MAGEA4 in Tumour Sample

1.1 PREPARATION OF FORMALIN-FIXED PARAFFIN-EMBEDDED (FFPE) TISSUE SAMPLES

Tissue samples were cut into approximately 5 mm slices and fixed for 24 hours (range 16-36 h) in a minimum volume of 10% NBF equal to 20× the tissue volume. The tissue specimens were no more than 5 mm depth in one of the dimensions to allow efficient perfusion of the formalin (e.g. 10 mm×10 mm×5 mm). If the sample was larger (e.g. 10 mm×10 mm×10 mm) then it was cut into two pieces (or more) so that, for each sample, one of the dimensions is 5 mm or less (e.g. cut into 2 samples each 10 mm×10 mm×5 mm). The tissue was removed from formalin after 24 hours, washed once in 70% ethanol and placed in a labelled tissue cassette. The sample cassette was loaded into the tissue processor and processed through a 3 wax tank program using the delayed start feature (program: 2×1 h room temperature 75% ethanol; 2×1 h room temperature 90% ethanol; 2×1 h room temperature 100% ethanol, 3×1 h room temperature xylene; 3×80 mins 60° C. wax).

The sample cassette was removed from the tissue processor when the program was completed and subsequently embedded in paraffin wax in an appropriate orientation (FFPE format). When fully solidified and cooled the wax blocks were stored at 4° C.

In the case that the staining assay is performed with archived FFPE samples, it is preferred that a quality check (QC) of the samples is performed to confirm that the samples have been suitable fixed and reduce false negatives. The QC check may be performed using an antibody against PTEN.

1.2 Staining Assay

The assay was performed using a Ventana BenchMark ULTRA Automated IHC/ISH slide staining system (Ventana Medical Systems Inc. USA), according to the following protocol.

Assay Reagents
Primary antibody—Mouse monoclonal anti-MAGE A4 antibody (Clone OTI1F9, Origene Cat #TA505362), diluted in blocking reagent to a concentration of 10 μg/ml (range 4-10 μg/ml)
EZ-Prep solution (Ventana, Cat #950-102) [aqueous detergent solution]
Cell conditioning fluid 1 (Ventana, Cat #950-124) (Tris-EDTA buffer, pH8-9)
Blocking reagent—diluent with casein (Ventana, Cat #760-219) (100 mM phosphate buffer with <20 mM proteins (casein and goat globulins), <50 mM salt, <15 mM EDTA, brij detergent and preservative (0.05% ProClin 300))
ultraView universal DAB detection kit (Ventana, Cat #760-500) comprising HRP-multimer—a cocktail of HRP labelled antibodies (goat anti-mouse IgG and IgM plus goat anti-rabbit) (<50 ug/ml); 3, 3′-diaminobenzidine tetrahydrochloride (DAB) chromogen (0.2%); hydrogen peroxide (0.04%) in phosphate buffer; copper sulphate (5 g/L) in acetate buffer).

Protocol

Slides were baked for 30 mins at 60° C. Deparaffinization was carried out by incubating the slides in reaction buffer for 3 cycles of 8 minutes at 69° C. Epitope retrieval was then performed at 95° C. for 20 mins in cell conditioning fluid 1. The slides were subsequently incubated in DAB inhibitor (component of ultraView detection kit) for 8 mins at room temperature, followed by incubation in blocking reagent for 12 mins at 36° C. Primary antibody was auto-dispensed at approximately 100 μl per slide and the slide incubated for 32 mins at 36° C. Antibody detection was performed using the ultraView detection kit as instructed by the manufacturer. The slides were incubated in HRP-multimer for 20 mins at 36°.

1.3 Slide Visualisation

On completion of the staining procedure, slides were washed briefly in wash buffer and rinsed with distilled water to remove residual oil. Slides were subsequently dehydrated (1×3 min 70% ethanol; 3×3 min 100% ethanol; 2×5 min 100% xylene) and mounted and cover-slipped with DPX on a CTM6 auto coverslip instrument. Slides were air-dried in the fume hood overnight

Slides were digitally imaged at 40× magnification on a 3DHistech Pannoramic250 scanner, calibrated weekly for white balance

A positive stain indicated the presence of MAGEA4 in the sample.

1.4 Results

Human tumour tissue sections were prepared and stained as described above. MAGEA4 expression in each FFPE sample was determined by qRT-PCT using standard methods and calculated as number of transcripts per 100 ng RNA.

FIG. 1 shows representative staining of samples obtained from lung, oesophageal, head and neck, and bladder cancers (A to D, respectively). In each case staining was carried out using an antibody concentration of 10 ug/ml. For each tumour type, staining intensity is shown to correlate with RNA levels (RNA-high, -medium or low/none).

As a control for the assay, healthy human tissue samples were obtained from testis (MAGEA4 positive) and ovary (MAGEA4 negative) and stained using the same procedure. Samples were obtained from 3 individual donors in each case.

FIG. 2 shows strong staining of testis samples and no staining of samples from ovary.

### Comparative Example 1

The human tumour samples used in Example 1 were stained using the same procedure except that the concentration of primary antibody was below the optimised range.

FIG. 3 shows a comparison of staining between optimised and non-optimised antibody concentrations (10 ug/ml and 1.25 ug/ml respectively), for four different tumours (A to D).

This example demonstrates that reducing the concentration of primary antibody results in sub-optimal staining. This could lead to false-negative results, particularly in samples with lower levels of MAGEA4.

1. A method of detecting MAGEA4 in a sample, the method comprising:
adding an anti-MAGEA4 antibody to the sample at a concentration in the range of from 2-20 μg/ml;
incubating the antibody and the sample; and
detecting antibody bound to the sample, wherein the antibody is OTI1F9.

2. The method of claim 1, wherein the concentration of the antibody is 4-15, 4-10, 5-13, 6-12 or 7-11 μg/ml.

3. The method of claim 2, wherein the concentration of the anti-MAGEA4 antibody is 10 μg/ml.

4. The method of any preceding claim, wherein the sample and the anti-MAGEA4 antibody are incubated for 30-60, 25-50, 25-39, 27-35, 28-34, 30-33, 31, 32 or 33 minutes and/or at 33-39, 34-38, 35-37, 35, 36 or 37° C.

5. The method of any preceding claim, wherein the sample and the anti-MAGEA4 antibody are incubated for 32 minutes at 36° C.

6. The method of any preceding claim, wherein anti-MAGEA4 antibody bound to the sample is detected indirectly by a secondary antibody that binds to the anti-MAGEA4 antibody.

7. The method of any preceding claim, wherein the secondary antibody is conjugated to horseradish peroxidase.

8. The method of any preceding claim, further comprising the step of epitope retrieval prior to adding the anti-MAGEA4 antibody to the sample.

9. The method of claim 8, wherein the temperature of the epitope retrieval step is 80-110, 90-100 or 92-108° C. and/or the duration of the epitope retrieval step is 10-30, 12-28, 13-27, 14-26, 15-25, 16-24, 17-23, 18-22 minutes.

10. The method of claim 8 or claim 9, wherein the step of epitope retrieval is performed at 95° C. for 20 minutes.

11. The method of any preceding claim, further comprising inhibiting endogenous peroxidase activity in the sample, optionally by contacting the sample with a DAB (3,3′-Diaminobenzidine) inhibitor.

12. The method of claim 11, wherein the sample is contacted with the DAB inhibitor for at least 5, 6, 7, 8, 9, 10 or 11 minutes, preferably, at least 8 minutes, at room temperature.

13. The method of any preceding claim, further comprising the step of contacting the sample with a blocking reagent, optionally after inhibiting endogenous peroxidase activity.

14. The method of claim 13, wherein the sample is contacted with the blocking reagent for 8-16, 9-15, 10-14 or 11-13 minutes and/or 33-39, 34-38, 35-37° C.

15. The method of claim 14, wherein the sample is contacted with the blocking reagent for 12 minutes at 36° C.

16. The method of any preceding claim, wherein the sample is embedded in paraffin

17. The method of claim 16, further comprising the step of deparaffinisation.

18. The method of any claim 17, wherein the step of deparaffinisation comprises incubating the sample in reaction buffer for 3 cycles of 8 minutes at 69° C.

19. The method of any preceding claim, comprising:
deparaffinising the sample, optionally by incubating the sample in reaction buffer such as EZ-Prep solution, optionally for 3 cycles of 8 minutes at 69° C.;
retrieving epitope, optionally by incubating the sample in cell conditioning fluid 1 at 95° C. for 20 mins;
inhibiting endogenous peroxidase activity, optionally by incubating the sample in DAB inhibitor for 8 mins at room temperature;
incubating the sample in blocking reagent, optionally for 12 mins at 36° C.;
adding antibody OFI1F9 to the sample at a concentration of 10 μg/ml;
incubating the antibody and the sample for 32 mins at 36° C.; and
detecting antibody that is bound to the sample.

20. A kit for detecting MAGEA4 in a sample, comprising an anti-MAGEA4 antibody in combination with the reagents necessary for carrying out the method of any one of claims 1 to 19.
